(19) United States
(12) Patent Application Publication
(10) Pub. No.: US 2007/0259844 A1
(43) Pub. Date: Nov. 8, 2007

(54) Antiviral Compositions And Methods Of Using The Same

(75) Inventor: Jong Joseph Kim, North Wales, PA (US)

Correspondence Address: COZEN O'CONNOR, P.C., 1900 MARKET STREET, PHILADELPHIA, PA, 19103-3508, US

(73) Assignee: VGX PHARMACEUTICALS, Blue Bell, PA (US)

(21) Appl. No.: 10/561,656

(22) PCT Filed: Jun. 21, 2004
(86) PCT No.: PCT/US04/19756

§ 371 Date: Nov. 17, 2006

Related U.S. Application Data
(60) Provisional application No. 60/480,499, filed on Jun. 20, 2003.

(51) Int. Cl. A61K 31/573; A61K 38/21; A61K 31/7056; A61K 31/551; A61K 31/522

(52) U.S. Cl. 514/179; 424/85.7; 514/43; 514/263.31; 514/220

(57) ABSTRACT

Pharmaceutical compositions for and methods of preventing infection or treating an individual who has been identified as being infected with a Flavivirus, Pestivirus, picornovirus or coronavirus such as HCV, GB virus B, JEV, WNV, CSFV, BDV, BVDV, poliovirus, FMDV, or EMCV, comprising administering to the individual a therapeutically effective amount of one or more glucocorticoid receptor antagonist compounds, wherein said compound has steroidal structure are disclosed.

### CROSS REFERANCE TO RELATED APPLICATIONS

This application claims the benefit of U.S. provisional Ser. No. 60/480,499, filed Jun. 20, 2003. The entire disclosures of this application is incorporated herein by reference.

### FIELD OF THE INVENTION

The present invention relates to compounds and compositions that have antiviral activity) and to the use of such compounds and compositions in methods of treating individual who have been exposed to, infected with viruses.

### BACKGROUND OF THE INVENTION

Eukaryotic cells and their viruses have evolved at least two mechanisms for recruiting and positioning ribosomes at the start sites for translation of RNA messages. The primary mechanism involves recognition of a 7-methyl guanosine cap on the 5′ terminus of the mRNA by a set of canonical initiation factors that recruit the 43S particle—including the 40S ribosomal subunit and eukaryotic initiation factor 3 (eIF3)—forming the 48S preinitiation complex (Merrick & Hershey, 1996; Pain, 1996; Sachs et al., 1997). Alternatively, numerous viruses and some eukaryotic mRNAs utilize a cap-independent pathway in which an RNA element, the internal ribosome entry site (IRES), drives preinitiation complex formation by positioning the ribosome on the message, either at or just upstream of the start site. In hepatitis C virus (HCV), the major infectious agent leading to non-A, non-B hepatitis, the minimum IRES includes nearly the entire 5′ untranslated region (UTR) of the message (for review, see Rijnbrand & Lemon, 2000). The secondary structure of the HCV IRES RNA, one of the most conserved regions of the entire viral genome, is critical for translation initiation, and is similar to that of the related pestiviruses and GB virus B (Brown et al., 1992; Wang et al., 1994, 1995; Le et al., 1995; Rijnbrand et al., 1995; Honda et al., 1996a, 1996b, 1999; Pickering et al., 1997; Varaklioti et al., 1998; Psaridi et al., 1999; Tang et al., 1999).

The 341-nucleotide 5′ non-translated region is the most conserved part of the hepatitis C virus (HCV) genome. It contains a highly structured internal ribosomal entry site (IRES) that mediates cap-independent initiation of translation of the viral polyprotein by a mechanism that is unprecedented in eukaryotes. The first step in translation initiation is assembly of eukaryotic initiation factor (eIF) 3, eIF2, GTP, initiator tRNA and a 40S ribosomal subunit into a 43S preinitiation complex (Buratti et al., 1998, Kieft et al., 2001). The HCV IRES recruits this complex and directs its precise attachment at the initiation codon to form a 48S complex in a process that does not involve eIFs 4A, 4B or 4F. The IRES contains sites that bind independently with the eIF3 and 40S subunit components of 43S complexes, and structural determinants that ensure the correct spatial orientation of these binding sites so that the 48S complex assembles precisely at the initiation codon.

HCV IRES RNA adopts a specific three-dimensional fold in the presence of physiological concentrations of metal ions (Kieftet al., 1999). Rather than forming a tightly packed globular structure, the RNA helices extend from two folded helical junctions, suggesting that the IRES RNA acts as a structural scaffold in which specifically placed recognition sites recruit the translational machinery. This is supported by the observation that eIF3 and the 40S ribosomal subunit, the two largest components of the 43S particle, bind directly to the HCV IRES RNA (Pestova et al., 1998). Unlike IRESs found in some other RNA viruses, such as poliovirus, the IRES RNA•40S•eIF3 ternary pre-initiation complex forms without the involvement of other cellular factors (Pestova et al., 1998). Although several other proteins appear to interact with the HCV IRES RNA, they are not required for 43 S binding to the IRES (Ali & Siddiqui, 1995, 1997; Yen et al., 1995; Hahmet al., 1998; Fukushi et al., 1999).

IRES/EIF/40S complexes have been reported to be important for other RNA viruses. Flavivruses (such as GB virus B, Japanese Encephalovirus (JEV) and West Nile Virus (WNV)] (Malancha & Sudhanshu, 2000, Blackwell & Brinton, 2000) as well as pestiviruses (such as classical swine fever virus (CSFV), border disease virus (BDV), and bovine viral disease virus (BVDV)) (Sizova et al, 1998, Pestova et al., 1998, Fletcher et al., 2002) and picomoviruses [such as poliovirus, Foot and mouth disease virus (FMDV), and encephalomyocarditis virus (EMCV)) (Jang et al., 1988, Pelletier & Sonenberg, 1988). Similar ribosomal binding sites on coronaviruses have been reported to be important for RNA translation, replication, or transcription (O'Connor & Brian, 2000, Raman et al, 2003).

Using a yeast two-hybrid system, the cDNA of a Vpr-interacting cellular factor, termed human Vpr Interacting Protein (hVIP/mov34) was cloned (Mahalingam et al., 1998) hVIP/mov34 has complete homology with a reported member of the eIF3 complex (Asano et al., 1997). eIF3 is a large multimeric complex that regulates transcriptional events and is essential for G1/S and G2/M phase progression through the cell cycle. hVIP is thought to be a GR-responsive protein. Experimental results strongly suggest that hVIP is associated with the activated glucocorticoid receptor complex.

Glucocorticoids regulate diverse functions and are important to maintain central nervous system, cardiovascular, metabolic, and immune homeostasis. They also exert anti-inflammatory and immunosuppressive effects, which have made them invaluable therapeutic agents in numerous diseases (Chrousos, 1995). The actions of these hormones are mediated by their specific intracellular receptors, such as the GR. Several host co-activators of the GR have been described that directly interact with GR and components of the transcription initiation complex to enhance the glucocorticoid signal to the transcription machinery (Shibata et al., 1997).

The GR is the prototypic member of the translocating class of steroid receptors that are ubiquitously expressed in almost all human tissues and organs. Unliganded GR is found in the cytoplasm and moves rapidly into the nucleus in response to hormone stimulation (Htun et al., 1996, McNally et al., 2000). GR interacts in the cytoplasm with a complex array of chaperone proteins, including HSP90 and HSP70, and ligand-dependent displacement of these proteins is thought to be intimately involved in the translocation process (Bamberger et al., 1996, Beato et al., 1996). Both GR and hVIP are known Vpr ligands. Steroid hormone receptor antagonists such as mifepristone prevent the GR from moving into the nucleus in response to appropriate stimulation. In addition, mifepristone blocks the Vpr-induced nuclear entry of hVIP.HVIP had been reported as a potential Vpr ligand and demonstrated its role in cell cycle regulation as antisense of this gene induced cell cycle arrest at the G2/M phase (Mahalingam et al., 1998).

Glucocorticoids have been demonstrated to mimic the effects of Vpr; mifepristone has been shown to revert these effects of Vpr (Ayyavoo et al., 1997, Ayyavoo et al., 2002, Kino et al., 1999, Sherman et al., 2000). Moreover, mifepristone has been shown to block the nuclear translocation of hVIP induced by Vpr in cells. This result clearly demonstrates that mifepristone inhibits the translocation of hVIP induced by the expression of Vpr.

Infection with HCV is a major cause of human liver disease throughout the world. HCV is most commonly transmitted via blood and is considered to be the most common causative agent of post-transfusion non-A, non-B hepatitis (NANBH). It is estimated that there are about 5 million HCV infected individuals in the U.S. and 200 million worldwide. While infected individuals are often non-symptomatic, the U.S. Center for Disease Control reports that 75 to 85% of HCV infected persons may develop long-term infection, 70% may develop chronic liver disease, 10-20% may develop cirrhosis over a period of 20 to 30 years, and 1-5% of persons may die from the consequences of long term infection (liver cancer or cirrhosis). Hepatitis C is a leading indication for liver transplants. Estimates for the treatment of HCV infection in the United States range form $1-5 billion per year; such costs, together with mortality rates, are expected to rise significantly as more and more infected individuals become symptomatic.

Currently, a 24- or 48-week course of the combination of pegylated alpha interferon and ribavirin is the standard treatment for hepatitis C. Alpha interferon is a protein with natural antiviral activity that is made by individuals in response to viral infections. Pegylated interferon, of Peginterferon, is alpha interferon that has been modified chemically by the addition of a large inert molecule of polyethylene glycol in order to change the uptake and distribution and extend half-life. Ribavirin is an oral antiviral agent that by itself has little effect on HCV, but dramatically increases response rate when given in combination with interferon therapy.

Many adverse side effects are associated with therapy (flu-like symptoms, leukopenia, thrombocytopenia, depression, anemia, etc.); only about 50-80% of the patients respond (reduction in serum HCV RNA levels, normalization of liver enzymes); however, of those treated, 50-70% relapse within 6 months of cessation of therapy.

HCV is a Flavivirus. Other Flavivruses include GB virus B, Japanese Encephalovirus (JEV) and West Nile Virus (WNV). Examples of Pestiviruses include classical swine fever virus (CSFV), border disease virus (BDV), and bovine viral disease virus (BVDV) and picomoviruses include poliovirus, Foot and mouth disease virus (FMDV), and encephalomyocarditis virus (EMCV)). Another viral pathogen family is the coronaviruses. Each of these viruses is pathogenic and share similar ribosomal binding sites important for RNA translation, replication, or transcription.

There remains a need to identify compounds which have antiviral activity that can be used in the treatment of virally infected patients.

### SUMMARY OF THE INVENTION

The present invention relates to methods of treating an individual who has been identified as being infected with a Flavivirus, Pestivirus, picornovirus or coronavirus, such as HCV, GB virus B, JEV, WNV, CSFV, BDV, BVDV, poliovirus, FMDV, or EMCV. The methods comprise administering to said individual a therapeutically effective amount of one or more glucocorticoid receptor antagonist compounds, wherein said compound has steroidal structure.

The present invention also relates to methods of preventing infection by a Flavivirus, Pestivirus, picornovirus or coronavirus, such as HCV, GB virus B, JEV, WNV, CSFV, BDV, BVDV, poliovirus, FMDV, or EMCV. The methods comprise administering to said individual a prophylactically effective amount of one or more glucocorticoid receptor antagonist compounds, wherein said compound has steroidal structure.

The present invention also relates to pharmaceutical compositions comprising one or more glucocorticoid receptor antagonist compounds having steroidal structure in a therapeutically effective amount to treat or prevent Flavivirus, Pestivirus, picomovirus or coronavirus infection, such as infection by HCV, GB virus B, JEV, WNV, CSFV, BDV, BVDV, poliovirus, FMDV, or EMCV.

### BRIEF DESCRIPTION OF THE FIGURES

FIG. 1 shows the steady-state concentration of mifepristone in the patient serum reported in published studies. In these clinical studies, the steady-state drug concentrations of 35-2300 ng/ml were achieve through daily doses of 1-200 mg.

FIG. 2 shows data from experiments described in Example 2 regarding anti-HCV activity of mifepristone.

### DETAILED DESCRIPTION OF PREFERRED EMBODIMENTS

The present invention arises out of the discovery that glucorticoid receptor antagonist compounds inhibit HCV replication. HCV RNA levels are reduced or eliminated by administration of glucorticoid receptor antagonist compounds to individuals infected with HCV. In individuals with hepatitis C, HCV RNA levels are reduced or eliminated and serum ALT levels improve following administration of glucorticoid receptor antagonist compounds. Treatment of HCV infected individuals, particularly those with hepatitis C symptoms may be by the administration of glucorticoid receptor antagonist compounds alone or in combination with other antiviral therapies such as alpha interferon, including Peginteferon, alone, or alpha interferon, including Peginteferon, together with Ribivirin.

While not wishing to be bound by theory, it is believed that the mechanism by which the present invention operates includes the disruption of the protein mov34, a member of EIF3 complex. EIF3/viral IRES/40S complex is important for viral protein translation of hepatitis C virus (HCV) and other viruses. GRII antagonist drugs, including Mifepristone, can target and disrupt function/structure of mov34, a member of EIF3 complex. Accordingly, drug compounds that block/inhibit EIF3/mov34 (antisense, antibodies, inhibitory RNA) can be used as a treatment for HCV infection. Accordingly, HCV infection may be treated by administering an individual identified as being exposed to or infected with HCV, a therapeutic amount of a compound that disrupts mov34. HCV-infected individual suffering from hepatitis C may be administered one or more compounds that disrupt mov34 in an amount effective to decrease or eliminate detectable amounts of HCV RNA. The compounds of the invention may act as steroid hormone receptor antagonists that interactively blocks Rip-1/mov34, alone or in association with one or more steroid receptors, or other components, or one or more steroid receptors alone, preventing or inhibiting formation and translocation of the Rip-1 and/or steroid receptor or other EIF component complex.

According to the present invention, glucocorticoid receptor antagonist compounds may be used to treat individuals at a high risk of having been exposed to or those diagnosed as having been infected with Flavivruses, Pestiviruses, picomoviruses and coronaviruses. Amounts of such compounds effective to inhibit viral replication are administered to such individuals. According to the present invention, glucocorticoid receptor antagonist compounds may be used to treat individuals exposed to or infected with HCV, GB virus B, JEV, WNV, CSFV, BDV, BVDV, poliovirus, FMDV, and EMCV.

Examples of antiviral compounds include glucocorticoid receptor type II antagonist compounds, particularly those having a steroidal structure. In accordance with the present invention, a preferred group of glucocorticoid receptor antagonists are those to which mifepristone, better known as RU-486, belongs. This compound, 11β-(4-dimethylaminophenyl)-17β.-hydroxy-17β.-(propyl-1-ynyl)estra-4,9-dien-3-one, is a good glucocorticoid antagonist, which also has antiprogestin activity. Further details concerning this and related compounds may be found in Agarwal, M. K. et al. “Glucocorticoid antagonists” FEBS LETTERS 217, 221-226 (1987), which is incorporated herein by reference. As noted, agonist activity requires an alcohol group at the 11β position. Accordingly, glucocorticoid receptor antagonists used in the present invention preferably are not hydroxylated at the 11β position. In preferred embodiments, the glucocorticoid receptor antagonists used in the present invention preferably are not hydroxylated at the 11β position and are resistant to reactions in which the 11β position becomes hydroxylated. Extensive work has been done over the years in synthesizing and testing glucocorticoid antagonists which belong to this group, and the published literature is an abundant guide for the selection of candidate compounds that fall within the scope of the present invention. The patent literature alone is substantial. Thus, reference is made to the following U.S. patents, all of which are incorporated herein by reference in their entirety, and particularly with respect to the descriptions of the compounds disclosed therein being described herein and their use in the treatment of individuals infected with virus as well as individuals suffering from viral disease, and the prevention of viral infection in individuals exposed to a virus: U.S. Pat. Nos. 4,296,206; 4,386,085; 4,447,424; 4,477,445; 4,519,946; 4,540,686; 4,547,493; 4,634,695; 4,634,696; 4,753,932; 4,774,236; 4,814,327; 4,829,060; 4,861,763; 4,912,097; 4,943,566; 4,954,490; 4,978,657; 5,006,518; 5,043,332; 5,064,822; 5,073,548; 5,089,488; 5,089,635; 5,093,507; 5,095,010; 5,095,129; 5,132,299; 5,166,146; and 5,276,023. Analysis of the patents set out above and the attendant technical literature reveals that the 11-position substituent, and particularly the size of that substituent, plays a key role in determining the antiglucocorticoid activity. The character of the A ring is also important. It is also noted that a 17-hydroxypropenyl side chain generally decreases antiglucocorticoidal activity in comparison to the 17-propinyl side chain containing compounds, and that generally 9α,10α-CH₂ groups decrease antiglucocorticoidal activity. According to preferred embodiments, the pharmaceutical compositions of the present invention comprise a compound having a structure selected from the group consisting of mifepristone, Compound D1, Compound D2, Compound D3, Compound D4, Compound D5, Compound D6, Compound D7, Compound D8, pharmaceutically acceptable salts thereof and combination thereof. D1 is compound Pregna-4,6-diene-3,20-dione. D1 is available as Sigma Product Number: R19,725-4 and MDL Number: MFCD00199858, and was described in GB 929271 and U.S. Pat. No. 3,362,968, which are each incorporated herein by reference. In some embodiments, the compounds described in GB 929271 and U.S. Pat. No. 3,362,968 are utilized. D2 is compound 17-α-ethynyl-17-β-hydroxyestr-5(10)-En-3-one. D2 is available as Sigma Product Number: R18,844-1 and MDL Number: MFCD00199015 and is described in U.S. Pat. No. 3,024,256, which is incorporated herein by reference. In some embodiments, the compounds described in GB 929271 and U.S. Pat. No. 3,024,256 are utilized. D3 is described in Indian patents, IN 33649 and IN 67932 and PCT publication WO92/19616, which are each incorporated herein by reference. In some embodiments, the compounds described in IN 33649 and IN 67932 and PCT publication WO92/19616 are utilized. D4 is described in JP 62012791 and WO01/01996, which are each incorporated herein by reference. In some embodiments, the compounds described in JP 62012791 and WO01/01996 are utilized. D5 is a combination of Hydrocortisone Acetate and Zidovudine. Hydrocortisone Acetate is available as Sigma Product Number: H4126; Zidovudine is available as Sigma Product Number: 11546.

The present invention provides methods of treating individuals who have been identified as being infected with a Flavivrus, Pestivirus, picomovirus or coronavirus, including HCV, GB virus B, JEV, WNV, CSFV, BDV, BVDV, poliovirus, FMDV, or EMCV, by administering to them a therapeutically effective amount of such compositions. The present invention further provides methods of preventing HCV infection in individuals exposed to Flavivruses, Pestiviruses, picomoviruses and coronaviruses, including HCV, GB virus B, JEV, WNV, CSFV, BDV, BVDV, poliovirus, FMDV, and EMCV, by administering to them a prophylactically effective amount of such compositions. The diagnosis of viral infection can be done routinely by those skilled in the art.

The present invention is useful to therapeutically treat an individual identified as infected with Flavivruses, Pestiviruses, picornoviruses and coronaviruses, including HCV, GB virus B, JEV, WNV, CSFV, BDV, BVDV, poliovirus, FMDV, and EMCV in order to eliminate, reduce or stabilize viral titer.

The present invention is useful to prophylactically treat a high risk individual from becoming infected with Flavivruses, Pestiviruses, picornoviruses and coronaviruses, including HCV, GB virus B, JEV, WNV, CSFV, BDV, BVDV, poliovirus, FMDV, and EMCV.

In individuals who have symptoms of hepatitis C such as low serum ALT levels, the treatment of the present invention may produce improvement is serum ALT levels.

As used herein, the term “high risk individual” is meant to refer to an individual who is suspected of having been exposed to a Flavivirus, Pestivirus, picornovirus or coronavirus, including HCV, GB virus B, JEV, WNV, CSFV, BDV, BVDV, poliovirus, FMDV, or EMCV. Such individuals include health care or other individuals who may have accidentally exchanged blood with a viral-infected individual, such as through an accidental needle stick, injuries that occur during emergency medical care, rescue or arrest and unprotected sexual contact. High risk individuals can be treated prophylactically before any detection of viral infection can be made.

As used herein, the term “therapeutically effective amount” is meant to refer to an amount of a compound which produces a medicinal effect observed as reduction or reverse in viral titer when a therapeutically effective amount of a compound is administered to an individual who is infected with a Flavivirus, Pestivirus, picomovirus or coronavirus, including HCV, GB virus B, JEV, WNV, CSFV, BDV, BVDV, poliovirus, FMDV, or EMCV. Viral titer may be inferred based upon HCV RNA levels whereby administration of a therapeutically effective amount of a compound to an individual results in a reduction or elimination of detectable virus. Therapeutically effective amounts are typically determined by the effect they have compared to the effect observed when a composition which includes no active ingredient is administered to a similarly situated individual. If two or more antiviral compounds are administered to an individual, a therapeutically effective amount is the amount of one compound required to produce a medicinal effect observed as reduction or reverse in viral titer in combination with the other antiviral compound or compounds in the amount so administered.

As used herein, the term “prophylactically effective amount” is meant to refer to an amount of a compound which produces a medicinal effect observed as the prevention of infection by a Flavivirus, Pestivirus, picomovirus or coronavirus, including HCV, GB virus B, JEV, WNV, CSFV, BDV, BVDV, poliovirus, FMDV, or EMCV in an individual when a prophylactically effective amount of a compound is administered to a high risk individual. Prophylactically effective amounts are typically determined by the effect they have compared to the effect observed when a composition which includes no active ingredient is administered to a similarly situated individual.

The invention provides novel pharmaceutical compositions comprising antiviral compounds that are inhibitors of viral replication. According to the invention, pharmaceutical compositions comprising glucocorticoid receptor antagonists, particularly steroidal compounds, preferably those that are not hydroxylated at position 11β, are useful as antiviral compounds that are inhibitors of viral replication such as replication of Flaviviruses, Pestiviruses, picornoviruses and coronaviruses, including HCV, GB virus B, JEV, WNV, CSFV, BDV, BVDV, poliovirus, FMDV, and EMCV. In preferred embodiments, the antiviral compounds included in the pharmaceutical compositions of the present invention have a formula selected from the group consisting of mifepristone, Compound D1, Compound D2, Compound D3, Compound D4, Compound D5, Compound D6, Compound D7, Compound D8 as set forth below, a pharmaceutically acceptable salt thereof or combinations thereof. The invention provides novel pharmaceutical compositions comprising antiviral compositions that inhibit viral replication.

In some embodiments the method of the invention additionally includes the use of the viral replication inhibitor compositions of the invention in combination with other methodologies to treat viral infection. In some embodiments, the viral replication inhibitor is administered in conjunction with other antiviral agents such as acyclovir, gancyclovir, foscamet, lamivudine, ribivirin, interferon alpha-2a, interferon alpha-2b, peginterferon alfa-2a, and peginterferon alfa-2b. In some embodiments the method of the invention additionally includes the use of the HCV replication inhibitor compositions of the invention in combination with other methodologies to treat Flavivruses, Pestiviruses, picomoviruses or coronaviruses, including HCV, GB virus B, JEV, WNV, CSFV, BDV, BVDV, poliovirus, FMDV, or EMCV infection.

The pharmaceutical compositions comprising HCV replication inhibitor compositions of the present invention may be administered by any means that enables the active agent to reach the agent's site of action in the body of the individual. Pharmaceutical compositions of the present invention may be administered by conventional routes of pharmaceutical administration. Pharmaceutical compositions may be administered parenterally, i.e. intravenous, subcutaneous, intramuscular, subdermally, transdermally. In some embodiments, the pharmaceutical compositions are administered orally. Pharmaceutical compositions are administered to the individual for a length of time effective to eliminate, reduce or stabilize viral titer. When used prophylactically, Pharmaceutical compositions are administered to the individual for a length of time during which monitoring for evidence of infection continues.

Pharmaceutical compositions of the present invention may be administered either as individual therapeutic agents or in combination with other therapeutic agents. They can be administered alone, but are generally administered with a pharmaceutical carrier selected on the basis of the chosen route of administration and standard pharmaceutical practice.

Dosage varies depending upon known factors such as the pharmacodynamic characteristics of the particular agent, and its mode and route of administration; age, health, and weight of the recipient; nature and extent of symptoms, kind of concurrent treatment, frequency of treatment, and the effect desired. Usually a daily dosage of active ingredient can be about 0.001 to 1 grams per kilogram of body weight, in some embodiments about 0.1 to 100 milligrams per kilogram of body weight. Ordinarily dosages are in the range of 0.5 to 50 milligrams per kilogram of body weight, and preferably 1 to 10 milligrams per kilogram per day. In some embodiments, the pharmaceutical compositions are given in divided doses 1 to 6 times a day or in sustained release form is effective to obtain desired results.

Dosage forms (composition) suitable for internal administration generally contain from about 1 milligram to about 500 milligrams of active ingredient per unit. In these pharmaceutical compositions the active ingredient will ordinarily be present in an amount of about 0.5-95 by weight based on the total weight of the composition. Generally, multiple administrations are performed. In some embodiments, dosage forms administered in a 24 hour period is less than 200 milligrams. In some embodiments, dosage forms administered in a 24 hour period is 150 milligrams or less. In some embodiments, dosage forms administered in a 24 hour period is 100 milligrams or less. In some embodiments, dosage forms administered in a 24 hour period is 75 milligrams or less. In some embodiments, dosage forms administered in a 24 hour period is 60 milligrams or less. In some embodiments, dosage forms administered in a 24 hour period is 50 milligrams or less. In some embodiments, dosage forms administered in a 24 hour period is 40 milligrams or less In some embodiments, dosage forms administered in a 24 hour period is 30 milligrams or less. In these pharmaceutical compositions the active ingredient will ordinarily be present in an amount of about 0.5-95 by weight based on the total weight of the composition. Generally, multiple administrations are performed.

According to preferred embodiments of the invention, the antiviral compounds are provided over a course of time in which a therapeutically effective amount of compound is present in the individual's body so as to reduce the viral titer to essentially undetectable levels or essentially undetectable levels such that an asymptomatic individual will not develop symptoms or the onset of such symptoms shall be delayed. According to such preferred embodiments, drug titer remains at antiviral levels in the individual who has been identified as being infected with the virus or who has a high likelihood of having been exposed to the virus for an extended period of time such as 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 or more days, 48 or more days, 60 or more days or 75 or more days. Mifeprestone has been used clinically and may have been administered to individuals infected with a Flavivirus, Pestivirus, picornovirus or coronavirus, including HCV, GB virus B, JEV, WNV, CSFV, BDV, BVDV, poliovirus, FMDV, or EMCV. Such coincidental administrations do not maintain the drug titer over a period long enough to provide therapeutic benefit sufficient to reduce viral titer enough to eliminate or reduce the onset of symptoms or reduce symptoms for a significant time.

Pharmaceutical compositions may be formulated by one having ordinary skill in the art with compositions selected depending upon the chosen mode of administration. Suitable pharmaceutical carriers are described in Remington's Pharmaceutical Sciences, A. Osol, a standard reference text in this field, which is incorporated herein by reference.

For parenteral administration, the compound can be formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques. In some embodiments, a parenteral composition suitable for administration by injection is prepared by dissolving 1.5% by weight of active ingredient in 0.9% sodium chloride solution.

According to some embodiments of the present invention, the composition is administered to tissue of an individual by topically or by lavage. The compounds may be formulated as a cream, ointment, salve, douche, suppository or solution for topical administration or irrigation. The compounds may be formulated as a transdermal patch or subdermal implants. Formulations for such routes administration of pharmaceutical compositions are well known. Generally, additives for isotonicity can include sodium chloride, dextrose, mannitol, sorbitol and lactose.

In some cases, isotonic solutions such as phosphate buffered saline are used. Stabilizers include gelatin and albumin. In some embodiments, a vasoconstriction agent is added to the formulation. The pharmaceutical preparations according to the present invention are preferably provided sterile and pyrogen free. The pharmaceutical preparations according to the present invention which are to be used as injectables are provided sterile, pyrogen free and particulate free.

A pharmaceutically acceptable formulation will provide the active ingredient(s) in proper physical form together with such excipients, diluents, stabilizers, preservatives and other ingredients as are appropriate to the nature and composition of the dosage form and the properties of the drug ingredient(s) in the formulation environment and drug delivery system.

In some embodiments, the invention relates to methods of treating patients suffering from HCV infection. In some embodiments, the invention relates to methods of preventing HCV infection in high risk individuals.

According to some embodiments of the invention, the patient is treated with other antiviral therapy in conjunction the administration of pharmaceutical compositions according to the invention. The use of multiple therapeutic approaches provides the patient with a broader based intervention.

According to some aspects of the present invention, in combination with administration of the composition that comprises the HCV replication inhibitor, the individual is also administered another agent. In some embodiments, in combination with administration of the composition, the individual additionally receives compositions that comprises acyclovir, ganciclovir, foscamet, lamivudine, ribavirin, interferon alpha-2a, interferon alpha-2b, peginterferon alfa-2a, and peginterferon alfa-2b.

Other antivirals may also be used delivered according to standard protocols using standard agents, dosages and regimens. In some embodiments, the pharmaceutical compositions contain one or more of the glucocorticoid receptor antagonist compounds. In some embodiments, the pharmaceutical compositions contain one or more of the compounds selected from the group consisting of mifepristone, Compound D1, Compound D2, Compound D3, Compound D4, Compound D5, Compound D6, Compound D7, Compound D8 pharmaceutically acceptable salts thereof and combination thereof. In some embodiments, the pharmaceutical compositions contain one or more glucocorticoid receptor antagonist compounds and at least one additional antiviral selected from the group consisting of: acyclovir, ganciclovir, foscarnet, lamivudine, ribavirin, interferon alpha-2a, interferon alpha-2b, peginterferon alfa-2a, and peginterferon alfa-2b, together with a pharmaceutically acceptable carrier. In some embodiments, the pharmaceutical compositions contain one or more of the compounds selected from the group consisting of mifepristone, Compound D1, Compound D2, Compound D3, Compound D4, Compound D5, Compound D6, Compound D7, Compound D8 pharmaceutically acceptable salts thereof and combination thereof and at least one additional antiviral selected from the group consisting of: acyclovir, ganciclovir, foscarnet, lamivudine, ribavirin, interferon alpha-2a, interferon alpha-2b, peginterferon alfa-2a, and peginterferon alfa-2b, together with a pharmaceutically acceptable carrier.

The pharmaceutical compositions according to the present invention may be administered as a single dose or in multiple doses. The pharmaceutical compositions of the present invention may be administered either as individual therapeutic agents or in combination with other therapeutic agents. The treatments of the present invention may be combined with conventional therapies, which may be administered sequentially or simultaneously.

In addition to treating virally-infected individual, the present invention relates to methods of preventing viral infection in high risk individuals who, for example, are suspected of having been exposed to the virus.

Additionally, the present invention is particularly useful to prevent recurrence of infection in patients who have been previously diagnosed as positive for a virus such as a Flavivirus, Pestivirus, picomovirus or coronavirus, including HCV, GB virus B, JEV, WNV, CSFV, BDV, BVDV, poliovims, FMDV, or EMCV, but show no indication of infection.

Those having ordinary skill in the art can readily identify high risk individuals. Healthcare workers come into contact with infected blood and suffer needle sticks from syringes used on virally infected individuals. Surgeons cut themselves during surgery. Lab workers, dentists and dental technicians come into contact with infected blood as do emergency medical and rescue workers and law enforcement officers. Individuals involved in athletics and sexually active individuals can also become exposed to the virus. Once any person comes into contact with infected blood, that individual is at an elevated risk of infection.

The present invention is not limited to any particular theory or mechanism of action and while it is currently believed that the compounds identified herein operate through blocking the steroid hormone receptor complex that comprises Rip-1/mov34, such explanation of the mechanism of action is not intended to limit the invention. The present invention is further illustrated by the following examples, which are not intended to be limiting in any way.

### EXAMPLES

### Example 1

Transdermal Drug Delivery

The skin is the largest and most accessible organ of the human body. The permeability of the skin and its ability to deliver drugs to the blood circulation makes it an ideal drug delivery route. Transdermal drug delivery is an increasingly, important method of drug administration. Transdermal drug delivery devices typically involve a carrier (such as a liquid, gel, or solid matrix, or a pressure sensitive adhesive) into which the drug to be delivered is incorporated. The drug-containing carrier is then placed on the skin and the drug, along with any adjuvants and excipients, is delivered to the skin.

Typically the portions of the carrier that are not in contact with the skin are covered by a backing. The backing serves to protect the carrier (and the components contained in the carrier, including the drug) from the environment and prevents loss of the ingredients of the drug delivery device to the environment. Backing materials that have found use in transdermal drug delivery devices include metal foils, metalized plastic films, and single layered and multilayered polymeric films.

Transdermal drug delivery utilizes the skin for the delivery of the drug molecules from the surface of the skin, through its layers, to the circulatory system. The transdermal drug delivery technology comprises of a controlling system that regulates the rate of drug delivery to the skin, and another that uses the skin to control the absorption rate.

Transdermal drug delivery occurs in two ways: passive and active transdermal delivery. Passive systems allow the drug to diffuse through the skin into the bloodstream using a simple concentration gradient as a driving force. Active delivery system requires a physical force to facilitate the movement of drug molecules across the skin.

The first transdermal patch was introduced in 1981. Subsequently, the applications of transdermal drug delivery have been expanded to include more products in multiple therapeutic areas. Numerous kinds of medications have been administered through the use of a patch, notably scopolamine for preventing motion sickness, nicotine derivatives intended to discourage an addicted smoker from continuing the smoking habit and estrogen hormones.

Prior art teaches us methods to load and deliver drugs via transdermal routes. U.S. Pat. No. 5,223,261 describes a loading and using a transdermal delivery system for delivering estradiol. U.S. Pat. No. 5,380,760 describes a transdermal delivery system for delivering prostaglandin. U.S. Pat. No. 5,702,720 describes a transdermal delivery system for delivering flurbiprofen. U.S. Pat. No. 6,132,760 describes a transdermal delivery system for delivering testosterone.

The amount of drug that constitutes a therapeutically effective amount varies according to the condition being treated, any drugs being coadministered with the drug, desired duration of treatment, the surface area and location of the skin over which the device is to be placed, and the selection of adjuvant and other components of the transdermal delivery device. Accordingly, it is not practical to enumerate particular preferred amounts but such can be readily determined by those skilled in the art with due consideration of these and other appropriate factors. Generally, however, the drug is present in the adhesive layer in an amount of about 2 to about 9 percent, preferably about 2.5 to about 6.5 percent, by weight based on the total weight of the adhesive layer. A device of the invention preferably contains a therapeutically effective amount of the drug dissolved in the adhesive layer.

The adhesive layer of the device of the invention also comprises one or more polymers, typically one or more copolymers. The polymer(s) utilized in the practice of the invention should be substantially chemically inert to the drug, and is preferably a pressure sensitive skin adhesive. Examples of suitable types of adhesives include acrylates, natural and synthetic rubbers, polysiloxanes, polyurethanes, and other pressure sensitive skin adhesives known in the art, either alone or in combination. Preferably the adhesive is an acrylate copolymer.

Delivery of Mifepristone/GR II Antagonists Via Transdermal Patch

The present invention provides transdermal drug delivery devices containing mifepristone, Compositions D1-D8 or other GRII antagonists (Drugs). The drug is present in the adhesive layer in a therapeutically effective amount, i.e., an amount effective to allow the device. to deliver sufficient amount of the drug to achieve a desired therapeutic result in the treatment of a condition.

A delivery of mifepristone via a transdermal patch would reduce the number of drugs a patient must take orally and improve compliance. The transdermal drug delivery would be most appropriate in cases where low systemic and steady state drug concentration is desirable. This delivery method could enhance patient compliance and could reduce the effects of potential drug toxicities.

There are several advantages of delivering anti-viral drugs via transdermal delivery systems. Transdermal drug delivery is not subjected to first-pass effect and does not cause frequent drug concentration alterations as compared to the drugs delivered through the oral route. This reduces the required dose in comparison to the oral drug delivery. Medications delivered via skin patches avoid liver metabolism and hence allow for lower doses of medication. It also avoids potential toxicity of the drug to the liver. The transdermal drug delivery also offers the flexibility of terminating the drug administration by simply removing the patch from the skin. This delivery system releases a controlled amount of a drug over a long period of time. Transdermal patch systems exhibit slow controlled drug release and absorption and the plasma drug concentration does not vary significantly over time. This delivery method would enhance patient compliance and thereby a reduction of drug resistant viruses as well as reduce the effects of potential drug toxicities.

Subdermial Drug Delivery (Implantable Devices)

A principal advantage of employing sustained-release compositions is that many therapeutic agents would otherwise be rapidly metabolized or cleared from the patient's system necessitating frequent administration of the drug to maintain a therapeutically effective concentration.

Accordingly, a variety of sustained release devices have been designed for oral, rectal and subcutaneous administration. “Matrix” type devices typically consist of an active compound dispersed in a matrix of carrier material which may be either porous or non-porous, solid or semi-solid, and permeable or impermeable to the active compound. These devices are rather easily prepared; however, they are not suitable for administering some pharmacologically active compounds. In addition, the rate of release of the active compound decreases with time. “Reservoir” type devices consist of a central reservoir of active compound surrounded by a rate controlling membrane (rcm). The rcm is generally a porous or a non-porous material which is non-biodegradable. In the case of the transdermal devices of this type, to maintain an effective concentration of active compound, the rate controlling membrane must have a large surface area. Thus, a common disadvantage of these devices is that their large size makes administration quite inconvenient. Other sustained release devices are hybrid-type devices which contain a matrix core surrounded by a rcm. Yet other devices are mechanical in nature, and include active compound-filled electrical or osmotic pumps.

The subdermally implantable devices of the present invention can be prepared in a variety of sizes and shapes to accommodate such factors as the specific implantation site and the desired release rate of the drug. In a preferred embodiment wherein the drug is a contraceptive agent, the device is substantially cylindrical in shape having a preferred overall length of from about 4.2 cm to about 4.6 cm, and a preferred overall diameter of from about 2.3 mm to about 2.7 mm. In such a case, the central core is rod-shaped, and has a preferred length of from about 3.8 cm to about 4.2 cm, and a preferred diameter of from about 2.0 mm to about 2.2 mm. These dimensions can be modified depending upon such factors as the implantation site and method of implantation, the subject, the condition to be treated, the drug, and the desired release rate of the drug, etc. For example, the length of the implantable device can be varied to deliver different amounts of the drug.

Prior art teaches us methods to load and deliver drugs via subdermal routes. The subdermally implantable devices according to the present invention can be easily fabricated in accordance with standard techniques. Once the drug is mixed with the matrix material to achieve a substantially uniform dispersion, the desired shape of the resultant dispersion is achieved by molding, casting extrusion, or other appropriate process. When the matrix material contains polymers such as silicone elastomers, an additional curing step may be necessary. The intermediate layer is then applied to the thus-shaped matrix, e.g., by swelling, coating or laminating according to known techniques, a polymeric tube in water and then placing it over the matrix and allowing the polymer to dry in place, or by mechanical lapping. The outer layer can likewise be applied in a variety of ways such as by mechanical stretching, swelling or dipping. See, for example, U.S. Pat. Nos. 3,832,252, 3,854,480 and 4,957,119. U.S. Pat. No. 5,756,115 describes a loading and using a subdermal delivery system for delivering contraceptives. The dimensions of the implant are also determined on the basis of the implantation method. The devices of the present invention can be implanted into a subject in accordance with standard procedures.

The present invention provides subdermal drug delivery devices containing mifepristone, Compositions D1-D8 or other GRII antagonists (Drugs). The drug is present in the implantable devices in a therapeutically effective amount, i.e., an amount effective to allow the device to deliver sufficient amount of the drug to achieve a desired therapeutic result in the treatment of a condition.

Sustained and Controlled Release Drug Delivery

To improve the effectiveness of drug therapy and to reduce possible systematic side effects, many attempts have been made to deliver drugs in a controlled profile to human patients. The advantages of controlled release dosage forms are well known in both the pharmaceutical and medical sciences. The therapeutic benefits of controlled-release dosage forms include the pharmacokinetic ability to maintain a preplanned blood level of an administered drug over a comparatively longer period of time. The therapeutic benefits include also a simultaneous increase in patient compliance and a reduction in the number of doses of drug administered to a patient.

The prior art made available controlled release dosage that sought to provide a drug release rate profile that matched the blood physiological and chrono-pharmacological requirements needed for therapy. For example, an osmotic dosage form for delivering various drugs to a patient environment of use is presented in U.S. Pat. No. 3,845,770 issued to patentees Theeuwes and Higuchi, and in U.S. Pat. No. 3,916,899 issued to the same patentees. The dosage forms disclosed in these patents are manufactured comprising a wall that surrounds a compartment comprising a drug with an exit in the wall for delivering the drug to a patient. In U.S. Pat. Nos. 4,008,719; 4,014,334; 4,058,122; 4,116,241; and 4,160,452 patentees Theeuwes and Ayer made available dosage forms comprising an inside and an outside wall made of poly(cellulose acylate) for delivering a dosage of drug to a patient in need thereof.

Additional semipermeable polymers comprise acetaldehyde dimethylcellulose acetate; cellulose acetate ethylcarbamate; cellulose acetate methylcarbamate; cellulose diacetate propylcarbamate; cellulose acetate diethylaminoacetate; ethyl acrylate methyl methacrylate, semipermeable polyamide; semipermeable polyurethane; semipermeable sulfonated polystyrene; semipermeable crosslinked selective polymer formed by the coprecipation of a polyanion and polycation, as disclosed in U.S. Pat. Nos. 3,173,876; 3,276,586; 3,541,005; 3,541,006 and 3,546,876; semipermeable polymers as disclosed by Loeb and Sourirajan in U.S. Pat. No. 3,133,132; semipermeable, lightly crosslinked polystyrenes; semipermeable crosslinked poly (sodium styrene sulfonate); semipermeable crosslinked poly (vinylbenzyltrimethyl ammonium chloride); and semipermeable polymers possessing a fluid permeability in the range of 2.5×10⁻⁸ to 5×10⁻² (cm²/hr multidot.atm), expressed per atmosphere of hydrostatic or osmotic pressure difference across the semipermeable exterior wall 12. The polymers are known to the polymer art in U.S. Pat. Nos. 3,845,770; 3,916,899 and 4,160,020; and in Handbook of Common Polymers, by Scott, J. R. and Roff, W. J. 1971, CRC Press, Cleveland, Ohio. Wall 12, in a present manufacture can be coated from a substantially single solvent system, such as acetone if coated from a solution, or water if coated as a dispersion.

The present invention provides delivery of mifepristone, Compositions D1-D8 or other GRII antagonists (Drugs) via a sustained release or controlled release delivery techniques.

### Example 2

### Effective Clinical Dosage for Mifepristone

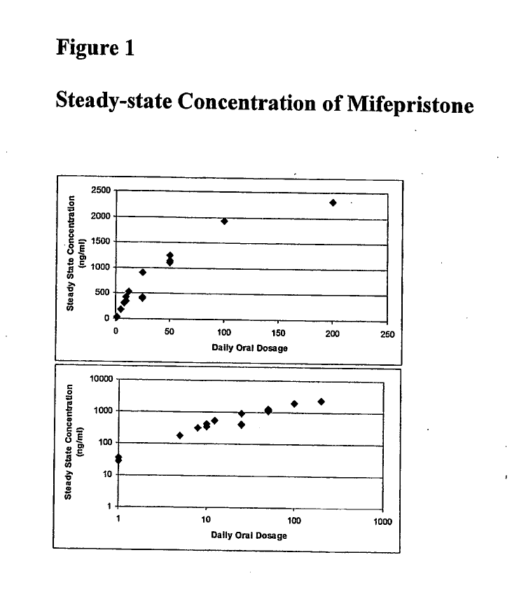

Mifepristone [11β-(4dimethylaminophenyl)-17β-hydroxy-17-α-(propyl-lynyl)-4,9-dien-3-one] is a glucocorticoid receptor antagonist with a molecular weight of 429.6 (C29H35NO2). Several studies have reported on the daily oral administration of mifepristone (multiple dosing) (Croxatto et al., 1992, Foldesi et al., 1996, Heikinheimo et al. 1989, 1993, Kekkonen et al., 1996, Sakar, 2002, Swahn et al., 1986). The steady-state concentrations of mifepristone in the patient serum reported in these studies are compiled in FIG. 1. In these clinical studies, the steady-state drug concentrations of 35-2300 ng/ml were achieve through daily doses of 1-200 mg (4-30 days).

### Example 3

### Primary In Vitro Anti-HCV Screen

The antiviral activity of test compounds are assayed in the stably HCV RNA-replicating cell line, AVA5, derived by transfection of the human hepatoblastoma cell line, Huh7 (Blight, et al., 2000, Sci. 290:1972). Compounds are added to dividing cultures once daily for three days (media is changed with each addition of compound). Cultures generally start the assay at 50% confluence and reach confluence during the last day of treatment. Intracellular HCV RNA levels and cytotoxicity are assessed 24 hours after the last dose of compound.

Assays are conducted using a single dose of test compound. Replicate cultures for HCV RNA levels (on 48-well plates) and triplicate cultures for cytoxicity (on 96-well plates) are used. A total of 3-4 untreated control cultures, and replicate cultures treated with 10 IU/ml α-interferon (the approximate EC₉₀ with no cytotoxicity), and 100 μM ribavirin (the approximate CC₉₀ with no antiviral activity) serve as positive antiviral and toxicity controls.

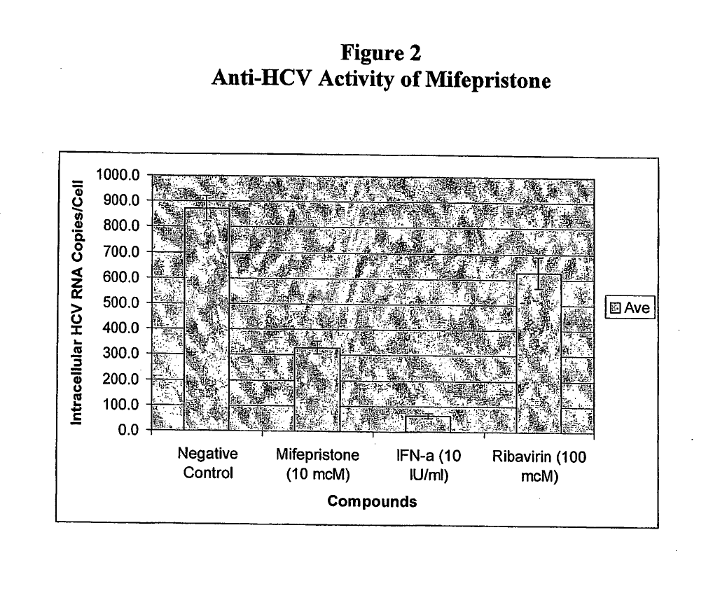

Intracellular HCV RNA levels are measured using standard blot hybridization (Korba & Gerin, 1992, Antivir. Res. 19:55) using triplicate cultures. HCV RNA levels in the treated cultures are expressed as a percentage of the mean levels of RNA detected in untreated cultures. Levels of β-actin RNA are used to normalize HCV RNA levels in each sample. Data is shown in FIG. 2.

### Example 4

### Mifepristone Metabolites

Unbound Mifepristone is metabolized by two-step demethylation or by hydroxylation, and the initial metabolic steps are catalysed by the cytochrome P450 (CYP) enzyme CYP3A4 (Jang et al., 1996 Biochem. Pharmacol. 52:753-761 and Reilly et al, 1999, which are incorporated herein by reference). Three metabolites of Mifepristone have been identified (Sarkar, 2002 Eur. J. of Obstetrics & Gynecol and Reprod. Biol. 101:113-120). This compound undergoes demethylation to produce mono-demethylated and di-demethylated derivatives as well as hydroxylation of the propynyl group to yield hydroxylated metabolite. Studies have shown that the metabolism of Mifepristone to mono-demethylated and hydroxylated metabolites was rapid but removal of the second methyl group leading to the formation of di-demethylated derivative occurred much more slowly and to much lesser extent than removal of the first. Serum levels of the monodemethylated metabolite always exceeded those of Mifepristone (Sarkar, 2002). The concentrations of the didemethylated and hydroxylated metabolites equalled or exceeded those of Mifepristone when the ingested dose was 400 mg or more. Monodemethylation and hydroxylation were rapid high-capacity reactions, whereas didemethylation was a lower-capacity reaction (Sarkar, 2002).

In each group of different dosage, positive correlations were found between the individual mean alpha 1-acid glycoprotein (AAG) concentrations and the peak concentration of Mifepristone measured at 1-2 h, versus the plateau concentration of Mifepristone measured at 6 h. The in-vitro studies showed that AAG was saturated by Mifepristone concentrations exceeding 2.5 microM. In serurn at 40 nM and 2.5 microM Mifepristone concentrations, 2.7% and 2.4%, respectively, of Mifepristone was not protein bound. These results suggest that AAG regulates in part the serum concentrations of Mifepristone, and Mifepristone exceeding the specific serum transport capacity is effectively metabolized.

Like Mifepristone, these metabolites are immunologically and biologically active and retain anti-progestational and anti-glucocorticoid properties. The relative binding affinities of the metabolites to the human glucocorticoid receptor are 61, 48 and 45% for the monodemethylated, hydroxylated, and didemethylated metabolites, respectively; each was higher than that of dexamethasone or cortisol (23%).

The data on Table 1 show comparative anti-progesterone, and anti-gluucocorticoid activities and the comparative ration of such activities for the mifepristone metabolites D6, D7 and D8. Mifepristone derivatives exhibiting reduced abortefaceint activity provide an advantage over mifepristone with respect to safety and elimination of side effects.

TABLE 1

| | Anti- | Anti- | |
|---|---|---|---|
| | Progesterone | Glucocorticoid | A-G/A-P |
| mono-demethylated | 21% | 61% | 290% |
| mifepristone | | | |
| metabolite D6 | | | |
| di-demethylated | 9% | 45% | 500% |
| mifepristone | | | |
| metabolite D7 | | | |
| hydroxylated | 15% | 48% | 320% |
| mifepristone | | | |
| metabolite D8 | | | |

Structures
D1: Pregna-4,6-diene-3,20-dione 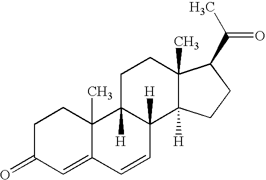
D2: 17-α-ethynyl-17-β-hydroxyestr-5 (10)-En-3-one 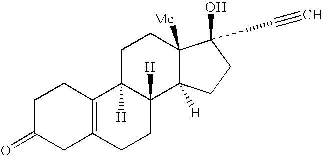
D3: Epoxyazadiradione 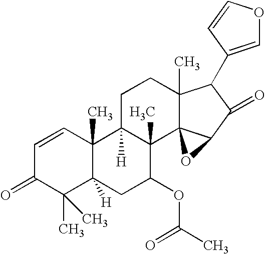
D4: NSC641295; Astragaloside II 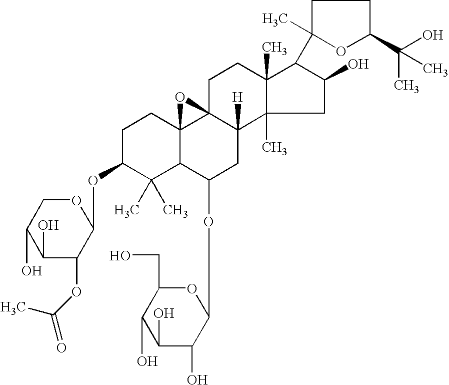
D5:: 3′Azido-3′deoxy-5′-O-[(11-.beta.-hydroxy-3-oxo-17-.beta.-androst-4-enyl)carbonyl]thymidine (Combination of Hydrocortisone Acetate and Zidovudine) 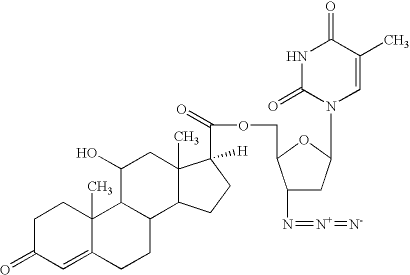
D6: mono-demethylated mifepristone metabolite 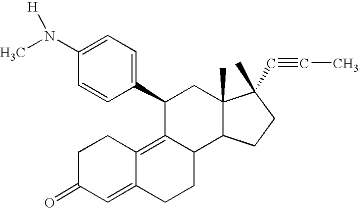
D7: di-demethylated mifepristone metabolite 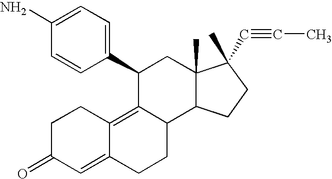
D8: hydroxylated mifepristone metabolite 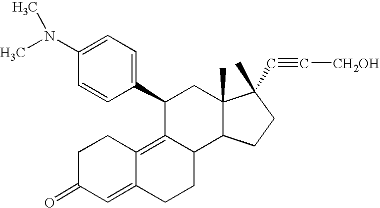
   Mifepristone

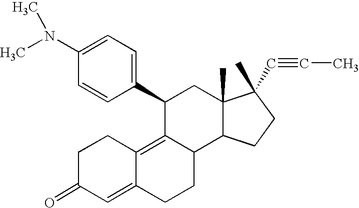

### REFERENCES

References cited herein including those set forth below are hereby incorporated herein by reference.

Ali N, Siddiqui A. 1995. Interaction of polypyrimidine tract-binding protein with the 59 noncoding region of the hepatitis C virus RNA genome and its functional requirement in internal initiation of translation. J Virol 69:6367-6375.

Ali N, Siddiqui A. 1997. The La antigen binds 59 noncoding region of the hepatitis C virus RNA in the context of the initiator AUG codon and stimulates internal ribosome entry site-mediated translation. Proc Natl Acad Sci USA 94:2249-2254.

Asano, K., Vornlocher, H. P., Richter-Cook, N. J., Merricj, W. C., Hinnebusch, A. G., and Hershey, W. B. (1997) J. Biol. Chem. 272, 27042-27052.

Ayyavoo, V., Mahboubi, A., Mahalingam, S., Ramalingam, R., Kudchodkar, S., Williams, W. V., Green, D. R., and Weiner, D. B. (1997) Nat. Med. 3, 1117-1123.

Ayyavoo, V., Muthumani, K., Kudchodkar, S. B., Zhang, D., Ramanathan, M. P., Dayes, N. S., Kim, J. J., Sin, J. I., Montaner, L. J., and Weiner, D. B. (2002) Int. Immunol. 14, 13-22.

Bamberger, C. M., Schulte, H. M., and Chrousos, G. P. (1996) Endocr. Rev. 17, 245-261.

Beato, M., and Sanchez-Pacheco, A. (1996) Endocr. Rev. 17, 587-609.

Blackwell, J L and Brinton, M A. 1997. Translation elongation factor-1alpha interacts with the 3′ stem-loop region of West Nile Virus genomic RNA. J Virol 71:6433-6444.

Brown E S, Zhang H, Ping L H, Lemon S M. 1992. Secondary structure of the 59 nontranslated regions of hepatitis C virus and pestivirus genomic RNAs. Nucleic Acids Res 20:5041-5045.

Buratti E, Tisminetzky S, Zotti M, Baralle F E. 1998. Functional analysis of the interaction between HCV 59 UTR and putative subunits of eukaryotic initiation factor eIF3. Nucleic Acids Res 26:3179-3187.

Chrousos, G. P. (1995) N. Engl. J. Med. 332, 1351-1362.

Croxatto, H B, Salvatierra A M, Croxatto, H D, Fuentealba B. Effects of continuous treatment with low dose mifepristone throughout one menstrual cycle. Human Reprod. 1992. 7:945-50.

Fletcher, S P et al. 2002. Pestivirus internal ribosome entry site (IRES) structure and function: Elements in the 5′ untranslated region important for IRES function. J Virol 76: 5024-5033.

Foldesi, I., G. Falkay and L. Kovacs, Determination of RU486 (mifepristone) in blood by radioreceptor assay: a pharmacokinetic study. Contraception 56 (1996), pp. 27-32.

Fukushi S, Okada M, Kageyama T, Hoshino F B, Katayama K. 1999. Specific interaction of a 25-kilodalton cellular protein, a 40S ribosomal subunit protein, with the internal ribosome entry site of hepatitis C virus genome. Virus Genes 19:153-161.

Heikinheimo, O., Kekkone R. Dose-response relationships of RU486. Ann Med. 1993. 25: 71-6.

Heikinheimo, O., Kontula K, H. Croxatto Spitz I, Luukkainen T, Lahteenmaki P Pharmacokinetics of the antiprogestin RU486 in women during multiple doses administration. J. Steroid Biochem 32 (1989), pp. 21-25.

Htun, H., Barsony, J., Renyi, I., Gould, D. L., and Hager, G. L. (1996) Proc. Natl. Acad. Sci. U.S.A. 93, 4845-4850.

Honda M, Beard M R, Ping L H, Lemon S M. 1999. A phylogenetically conserved stem-loop structure at the 59 border of the internal ribosome entry site of hepatitis C virus is required for cap-independent viral translation. J Virol 73:1165-1174.

Honda M, Brown E A, Lemon S M. 1996a. Stability of a stem-loop involving the initiator AUG controls the efficiency of internal initiation of translation on hepatitis C virus RNA. RNA 2:955-968.

Honda M, Ping L H, Rijnbrand R C, Amphiett E, Clarke B, Rowlands D, Lemon S M. 1996b. Structural requirements for initiation of translation by internal ribosome entry within genome-length hepatitis C virus RNA. Virology 222:31-42.

Jang et al. 1988. A segment of the 5′ nontranslated region of encephalomyocarditis virus RNA directs internal entry of ribosomes during in vitro translation, J. Virol. 62: 2636-2643.

Kekkonen, R., O. Heikinheimo E. Mandelin and P. Lahteenmaki, Pharmacokinetics of mifepristone after low oral doses. Contraception 54 (1996), pp. 229-234.

Kino, T., Gragerov, A., Kopp, J. B., Stauber, R. H., Pavlakis, G. N., and Chrousos, G. P. (1999) J. Exp. Med. 189, 51-62.

Kieft J S, Zhou K, Jubin R, Murray M G, Lau J Y, Doudna J A. 1999. The hepatitis C virus internal ribosome entry site adopts an ion-dependent tertiary fold. J Mol Biol 292:513-529.

Le S Y, Sonenberg N, Maizel J V Jr. 1995. Unusual folded regions and ribosome landing pad within hepatitis C virus and pestivirus RNAs. Gene 154:137-143.

Mahalingam, S., Ayyavoo, V., Patel, M., Kieber-Emmons, T., Kao, G. D., Muschel, R. J., and Weiner, D. B. (1998) Proc. Natl. Acad. Sci. U.S.A. 98, 3419-3424.

Malancha, T A, and Sudhanshu, V., 2000. Mov34 protein from mouse brain interacts with the 3′ noncoding region of Japanese encephalitis virus. J Virol 74: 5108-5115.

McNally, J. G., Muller, W. G., Walker, D., Wolford, R., and Hager, G. L. (2000) Science 287, 1262-1265.

Kieft J S, Zhou K, Jubin R, Doudna J A. 2001. Mechanism of ribosome recruitment by hepatitis C virus IRES. RNA. 7:194-206.

O'Connor J B, Brian D A 2000. Virology. 269:172-82.

Pestova T V, Shatsky I N, Fletcher S P, Jackson R J, Hellen CUT. 1998. Aprokaryotic-like mode of cytoplasmic eukaryotic ribosome binding to the initiation codon during internal translation initiation of hepatitis C and classical swine fever virus RNAs. Genes & Dev 12:67-83.

Pelletier, J., Sonenberg, N. 1988. Internal initiation of translation of eukaryotic mRNA directed by a sequence derived from poliovirus RNA. Nature. 334: 320-325.

Pickering J M, Thomas H C, Karayiannis P. 1997. Predicted secondary structure of the hepatitis G virus and GB virus-A 59 untranslated regions consistent with an internal ribosome entry site. J Viral Hepat 4:175-184.

Psaridi L, Georgopoulou U, Varaklioti A, Mavromara P. 1999. Mutationalanalysis of a conserved tetraloop in the 59 untranslated region of hepatitis C virus identifies a novel RNA element essential for the internal ribosome entry site fuiction. FEBS Lett 453:49-53.

Raman S, Bouma P, Williams G D, Brian D A. 2003. J Virol. 77:6720-30.

Rijnbrand R, Bredenbeek P, van der Straaten T, Whetter L, Inchauspe G, Lemon S, Spaan W. 1995. Almost the entire 59 non-translated region of hepatitis C virus is required for cap-independent trans-lation. FEBS Lett 365:115-119.

Rijnbrand R C, Lemon S M. 2000. Internal ribosome entry site-mediated translation in hepatitis C virus replication. Curr Top Microbiol Immunol 242:85-116.

Sarkar, N. N. Mifepristone: bioavailability, pharmacokinetics and use-effectiveness. 2002. Euro J Obst Gyneco and Repro Bio. 101:113-120.

Sherman, M. P., de Noronha, C. M., Pearce, D., and Greene, W. C. (2000) J. Virol. 2000 74, 8159-8165. Shibata, H., Spencer, T. E., Onate, T. E., Genster, S. Y., Tsai, S. Y., Tsai, M. J., and O'Malley, B. W. (1997) Recent Prog. Horm. Res. 52, 141-164.

Sizova D V, Kolupaeva V G, Pestova T V, Shatsky I N, Hellen CUT. 1998. Specific interaction of eukaryotic translation initiation factor 3 with the 59 nontranslated regions of hepatitis C virus and classicals wine fever virus RNAs. J Virol 72:4775-4782.

Swahn, M. L., G. Wang, A. R. Aedo, S. Z. Cekan and M. Bygdeman, Plasma levels of anti progestin RU486 following oral administration to non-pregnant and early pregnant women. Contraception 34 (1986), pp. 469-481.

Tang S, Collier A J, Elliott R M. 1999. Alterations to both the primary and predicted secondary structure of stem-loop IlIc of the hepatitis C virus 1b 59 untranslated region (59UTR) lead to mutants severely defective in translation which cannot be complemented in transby the wild-type 59UTR sequence. J Virol 73:2359-2364.

Varaklioti A, Georgopoulou U, Kakkanas A, Psaridi L, Serwe M, Casel-mann W H, Mavromara P. 1998. Mutational analysis of two un-structured domains of the 59 untranslated region of HCV RNA. Biochem Biophys Res Commun 253:678-685.

Wang C, Le S Y, Ali N, Siddiqui A. 1995. An RNA pseudoknot is an essential structural element of the internal ribosome entry site located within the hepatitis C virus 59 noncoding region. RNA1:526-537.

Wang C, Sarnow P, Siddiqui A. 1994. A conserved helical element is essential for internal initiation of translation of hepatitis C virus RNA. J Virol 68:7301-7307.

Yen J H, Chang S C, Hu C R, Chu S C, Lin S S, Hsieh Y S, Chang M F. 1995. Cellular proteins specifically bind to the 59-noncoding region of hepatitis C Virus RNA. Virology 208:723-732. 206 J. S. Kieft et al.

1. A method of treating an individual who has been identified as being infected with a Flavivirus, Pestivirus, picornovirus or coronavirus, comprising administering to said individual a therapeutically effective amount of one or more glucocorticoid receptor antagonist compounds, wherein said compound has steroidal structure.

2. The method of claim 1 wherein said compound has a steroidal structure free of a hydroxylated group at position 11β.

3. The method of claims 1 wherein the individual who has been identified as being infected with a HCV, GB virus B, JEV, WNV, CSFV, BDV, BVDV, poliovirus, FMDV, or EMCV

4. The method of claims 1 wherein a glucocorticoid receptor antagonist compound selected from the group consisting of: mifepristone, Compound D1, Compound D2, Compound D3, Compound D4, Compound D5, Compound D6, Compound D7, Compound D8, pharmaceutically acceptable salts thereof and combinations thereof is administered to said individual.

5. The method of claims 1 wherein mifepristone is administered to said individual.

6. The method of any one of claims 1 wherein one or more antiviral compounds selected from the group consisting of: acyclovir, ganciclovir, foscarnet, lamivudine, ribavirin, interferon alpha-2a, interferon alpha-2b, peginterferon alfa-2a, and peginterferon alfa-2b thereof is administered to said individual.

7. The method of claims 1 wherein the individual who has been identified as being infected with a HCV.

8. A method of preventing infection by a Flavivirus, Pestivirus, picornovirus or coronavirus in an individual suspected of having been exposed to such a virus comprising administering to said individual a prophylactically effective amount of one or more glucocorticoid receptor antagonist compounds, wherein said compound has steroidal structure

9. The method of claim 8 wherein said compound has a steroidal structure free of a hydroxylated group at position 11β

10. The method of claims 8 wherein the virus is HCV, GB virus B, JEV, WNV, CSFV, BDV, BVDV, poliovirus, FMDV, or EMCV.

11. The method of claims 8 wherein a glucocorticoid receptor antagonist compound selected from the group consisting of: mifepristone, Compound D1, Compound D2, Compound D3, Compound D4, Compound D5, Compound D6, Compound D7, Compound D, pharmaceutically acceptable salts thereof and combinations thereof is administered to said individual.

12. The method of claims 8 wherein mifepristone is administered to said individual.

13. The method of any one of claims 8 wherein one or more antiviral compounds selected from the group consisting of: acyclovir, ganciclovir, foscamet, lamivudine, ribavirin, interferon alpha-2a, interferon alpha-2b, peginterferon alfa-2a, and peginterferon alfa-2b thereof is administered to said individual.

14. The method of any one of claims 8 wherein the virus is HCV.

15. A pharmaceutical composition comprising one or more glucocorticoid receptor antagonist compounds having a steroidal structure in a therapeutically effective amount to treat or prevent Flavivirus, Pestivirus, picornovirus or coronavirus infection.

16. The pharmaceutical composition of claim 15 wherein said compound having a steroidal structure is free of a hydroxylated group at position 11β

17. The pharmaceutical composition of claims 15 in a therapeutically effective amount to treat or prevent HCV, GB virus B, JEV, WNV, CSFV, BDV, BVDV, poliovirus, FMDV, or EMCV infection.

18. The pharmaceutical composition of any one of claims 15 comprising a glucocorticoid receptor antagonist compound selected from the group consisting of: mifepristone, Compound D1, Compound D2, Compound D3, Compound D4, Compound D5, Compound D6, Compound D7, Compound D8, pharmaceutically acceptable salts thereof and combinations thereof.

19. The pharmaceutical composition of claims 15 comprising mifepristone.

20. The pharmaceutical composition of any one of claims 15 further comprising one or more antiviral compounds selected from the group consisting of: acyclovir, ganciclovir, foscarnet, lamivudine, ribavirin, interferon alpha-2a, interferon alpha-2b, peginterferon alfa-2a, and peginterferon alfa-2b.

21. The pharmaceutical composition of claims 15 in a therapeutically effective amount to treat or prevent HCV infection.
